(19) United States
(12) Patent Application Publication
(10) Pub. No.: US 2012/0123312 A1
(43) Pub. Date: May 17, 2012

(54) FULL COVERAGE ADHESIVE PATCH

(76) Inventor: Chi-Jung TAI, Kaohsiung City (TW)

(21) Appl. No.: 12/945,191

(22) Filed: Nov. 12, 2010

(51) Int. Cl. A61F 13/02

(52) U.S. Cl. 602/54

(57) ABSTRACT

A full coverage adhesive patch includes a base layer with an extended portion at one side, a first sticking portion and a second sticking portion formed respectively on the circumferential surface of the base layer and top edge of the extended portion, and a cover layer located on the base layer and extended portion. The cover layer is formed at a width mating surface width and height of two lateral sides of a user's finger or toe so that can fully wrap the finger or toe in an annular fashion. When the first and second sticking portions are adhered to the finger or toe, the patch of the invention can provide full coverage and waterproof protection for a wound. The cover layer has parallel first folding line and second folding line and mating first notch and second notch corresponding to the base layer and extended portion to enhance wrapping comfort.

### FIELD OF THE INVENTION

The present invention relates to a medical adhesive patch and particularly to an adhesive patch to fully cover the wounds on user's fingers or toes.

### BACKGROUND OF THE INVENTION

When people suffer injuries or perform a minor surgery, the wound usually is covered by a medical adhesive patch, such as a roll of ventilation adhesive tape, band-aid or tender wet, to fix the wound and prevent the wound from being infected and contaminated.

Refer to FIG. 1 for a conventional T-shaped band-aid. It adopts a two-layer structure, which includes a base layer 1A with a protrusion 2A extended from a transverse side to form a T-shaped fashion and a cover layer 3A laid on the base layer 1A and extended along the protrusion 2A to suit wrapping of user's wounded finger tip or toe tip. Also referring to FIGS. 2A and 2B, as the cover layer 3A usually is formed at a shorter length and narrower width, and does not fully cover the protrusion 2A and is spaced from lateral edges, when the wound 41A is located at a lateral side of the tip of a finger 4A, the cover layer 3A cannot fully bond and cover the wound. Or in the event that the wound is remote from the fingertip, or formed in a larger area, or located at the finger root, the cover layer 3A cannot provide enough protection for the wound. Referring to FIG. 2C, the cover layer 3A also can merely cover the front and rear surfaces of the finger 4A but cannot cover the entire finger 4A in an annular fashion. Hence the wound 41A on the lateral side of the fingertip is difficult to be wrapped and protected by the cover layer 3A of the conventional T-shaped band-aid. Moreover, the base layer 1A bonded to the wound 41A makes the finger 4A hard to stretch or bend, which is uncomfortable when in use. All these indicate that there is plenty of room for improvement.

### SUMMARY OF THE INVENTION

The primary object of the present invention is to provide a full coverage adhesive patch to overcome the shortcomings of the conventional techniques.

The full coverage adhesive patch according to the invention includes a cover layer, a base layer with an extended portion extended from one side thereof, a first sticking portion formed on the surface of the circumference of the base layer, and a second sticking portion at the top edge of the extended portion. The cover layer on the extended portion is formed at a width mating the surface width of user's finger or toe, and the cover layer on the base layer is formed at a width mating the height of two lateral sides of the finger or toe so that when in use the patch can fully wrap user's finger or toe in an annular fashion wherever wounds are located. Thus it can overcome the width or length constraint of the conventional band-aid that can merely cover a wounded area at the finger tip or toe tip.

Another object of the invention is to provide a full coverage adhesive patch having a first sticking portion and a second sticking portion located respectively on the circumference of the base layer and the top edge of the extended portion to form secure wrapping and full coverage of user's finger or toe, and provide desired waterproof and protection for the wounded portion.

To achieve the foregoing objects, the invention comprises a base layer with an extended portion extended from one side thereof, a first sticking portion formed on the surface of the circumference of the base layer and a second sticking portion at the top edge of the extended portion, and a cover layer laid on the base layer and extended portion and formed at widths mating respectively surface width and lateral height of user's finger or toe, such that the cover layer can fully wrap the user's finger or toe in an annular manner. Also the first and second sticking portions can fully cover wounded portion and provide waterproof effect. The cover layer further has a first folding line and a second folding line parallel with each other, and a first notch and a second notch that are corresponding respectively to the base layer and extended portion to improve wrapping snugness when in use.

The foregoing, as well as additional objects, features and advantages of the invention will be more readily apparent from the following detailed description, which proceeds with reference to the accompanying drawings.

### BRIEF DESCRIPTION OF THE DRAWINGS

FIG. 1 is a schematic view of a conventional T-shaped band-aid.

FIG. 2A is a schematic view showing wrapping of a user's finger of a conventional T-shaped band-aid.

FIG. 2B is another schematic view showing wrapping of a user's finger of a conventional T-shaped band-aid.

FIG. 2C is a sectional view taken on line 2C-2C in FIG. 2B.

FIG. 3 is an exploded view of the invention.

FIG. 4 is a perspective view of the invention.

FIG. 5A is a schematic view showing wrapping of a user's finger of the invention in a first using condition.

FIG. 5B is a schematic view showing wrapping of a user's finger of the invention in a second using condition.

FIG. 5C is a schematic view showing wrapping of a user's finger of the invention in a third using condition.

FIG. 5D is a sectional view taken on line 5D-5D in FIG. 5C.

FIG. 6 is a front view of an embodiment of the invention.

FIG. 7 is a front view of another embodiment of the invention.

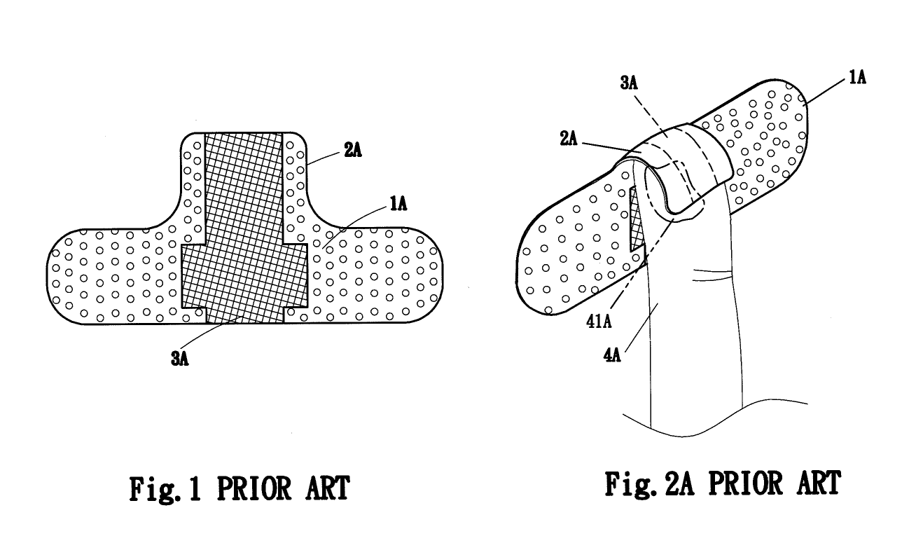

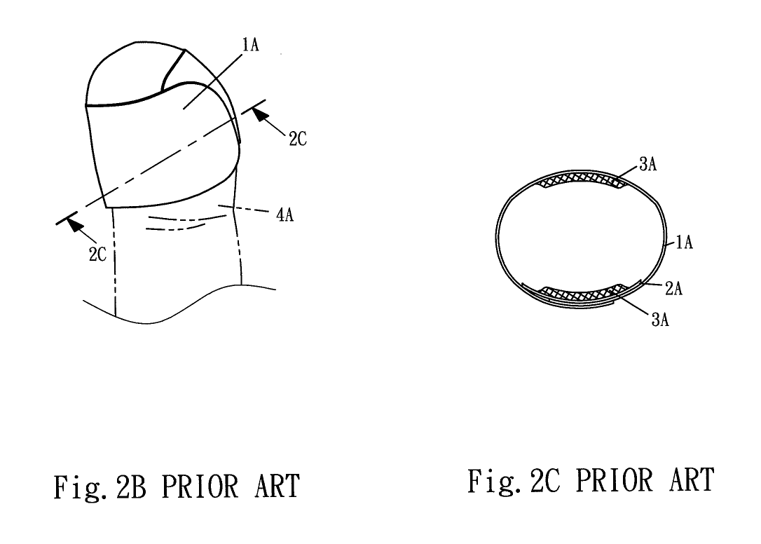

### DETAILED DESCRIPTION OF THE PREFERRED EMBODIMENTS

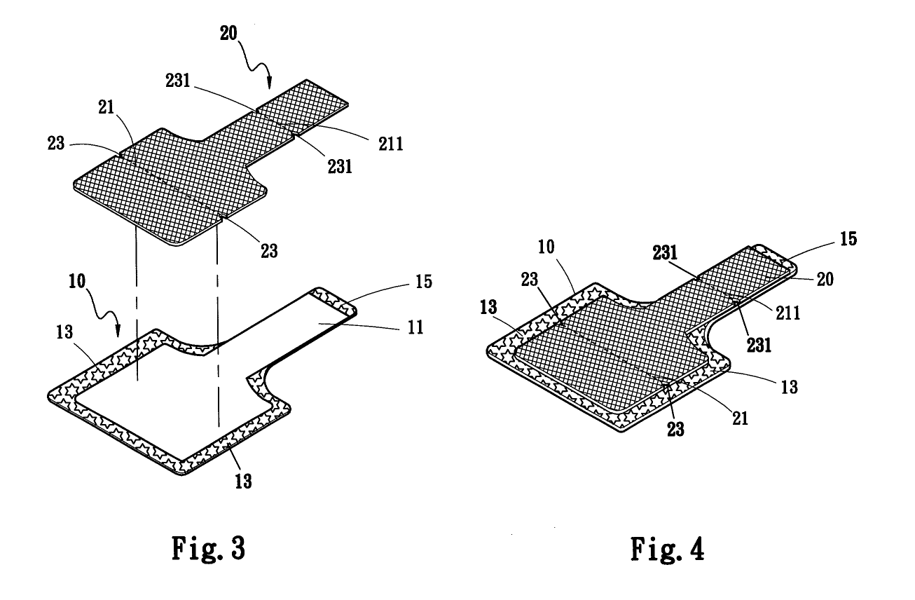

Please refer to FIGS. 3 and 4, the present invention aims to provide a full coverage adhesive patch which comprises a base layer 10 with an extended portion 11 extended from one side thereof, a first sticking portion 13 formed on the surface of the circumference of the base layer 10, a second sticking portion 15 at the top edge of the extended portion 11, and a cover layer 20 laid on the base layer 10 and extended portion 11 on the same surface where the first and second sticking portions 13 and 15 are located. The cover layer 20 is formed in a shape smaller than the profiles of the base layer 10 and extended portion 11.

The cover layer 20 on the extended portion 11 is formed at a width mating surface width of a user's finger or toe when in use. The cover layer 20 on the base layer 10 is formed at a width mating the height of two lateral sides of the finger or toe so that when in use the patch can fully cover user's finger or toe in an annular fashion. The cover layer 20 can be a gauze or non-woven fabric medicated with medicine or other therapeutic material.

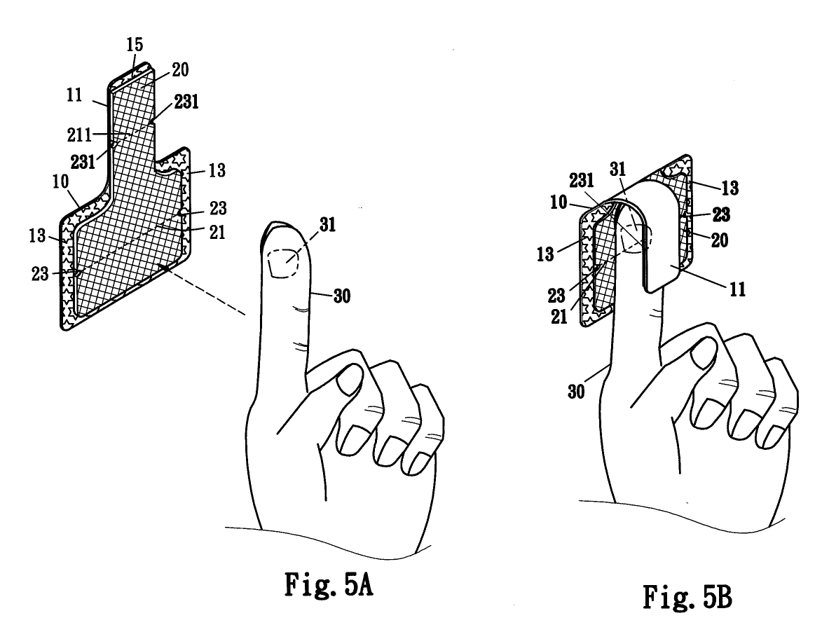

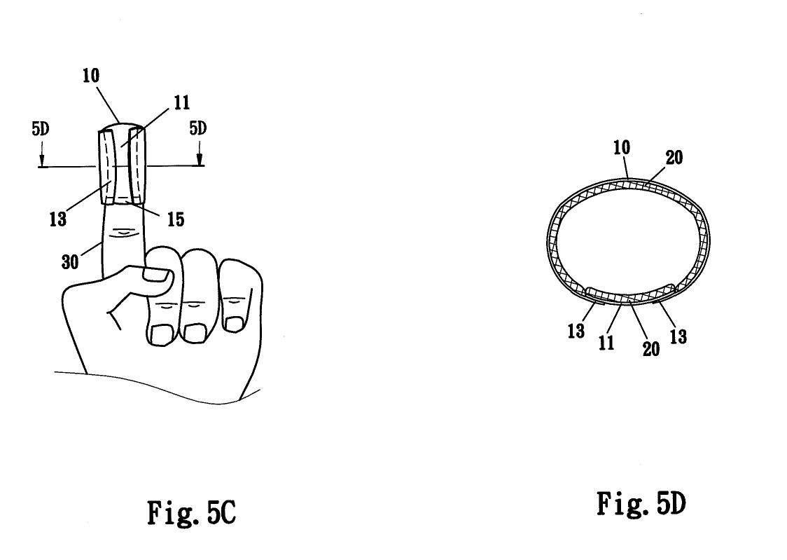

The cover layer 20 has at least one first folding line 21 and a second folding line 211 corresponding respectively to the center of the base layer 10 and extended portion 11 and parallel with each other, and a first notch 23 and a second notch 231 at each of two edges corresponding to the first folding line 21 and second folding line 211. The first and second folding lines 21 and 211 and the first and second notches 23 and 231 corresponding to user's finger or toe knuckles can facilitate bending or stretching when in use (referring to FIGS. 5A and 5B).

The first and second sticking portions 13 and 15 may be attached and covered by a removable protective layer (not shown in the drawings) on the surface thereof, which can be a release paper, release non-woven fabric, transparent plastic film or the like. The protective layer, in addition to on the first and second sticking portions 13 and 15, may also be enlarged to become an entire sheet mating the base layer 10 and extended portion 11 to protect the cover layer 20 from being contaminated. Or the invention may be held in a sealing bag (not shown in the drawings). When in use, the sealing bag is torn to take the patch of the invention out. Such a package can protect the cover layer 20 from being exposed to ambient air without being contaminated and impairing therapeutic effect.

Refer to FIGS. 5A through 5D for wrapping of a finger 30 as an example in the invention. The finger 30 has a wound 31 at the front edge and lateral side of the tip thereof. When in use, first, fetch the patch of the invention and peel off the protective layer (not shown in the drawings) on the first sticking portion 13 and second sticking portion 15, and place the patch on the finger 30 with the finger tip at the junction of the extended portion 11, base layer 10 and cover layer 20. At that time, the finger 30 is positioned in the center of the base layer 10 and the base layer 10 is at the left and right sides of the finger 30, such that the first folding line 21 and first notch 23 are corresponding to the knuckle of the finger 30.

For wrapping, first, bend the extended portion 11 downwards to allow the second sticking portion 15 to adhere to the inner side of the finger 30 with the second folding line 211 and second notch 231 being aligned to the knuckle of the finger 30; the width of the cover layer 20 on the extended portion 11 can match the surface width of the finger 30, and the width of the cover layer 20 on the base layer 10 can mate the height of the lateral sides of the finger 30, hence the wound 31 can be fully covered by the cover layer 20. Then the base layer 10 at two sides of the finger 30 can be bent towards the lateral sides of the finger 30, and finally the first sticking portion 13 is adhered to the inner side of the finger 30 with another side also being adhered thereto, thus is completed wrapping as shown in FIG. 5C. FIG. 5D shows the cross section of the finger 30 wrapped by the cover layer 20 in an annular fashion after wrapping is finished. Thus the invention can fully wrap and cover the external wounded portion such as skin disease, burn or scald on fingers or toes, or any other body parts.

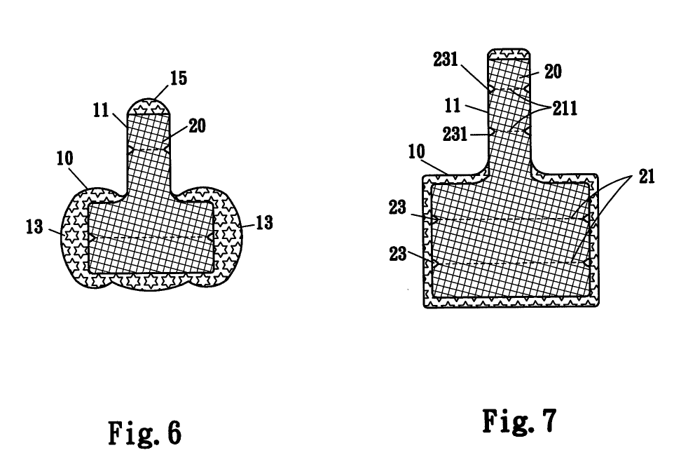

Refer to FIG. 6 for an embodiment of the invention in which the base layer 10, extended portion 11 and cover layer 20 can be flexibly designed and formed in varying types of shape according to requirements or ergonomics. The base layer 10 and cover layer 20 can be formed in a symmetrical arched shape corresponding to the shape of user's finger to make wrapping comfortably. When in use, the process is substantially the same as the embodiment previously discussed, hence details are omitted. In addition, the first and second sticking portions 13 and 15 can also be designed in different shapes to mate the profiles of the base layer 10, extended portion 11 and cover layer 20.

Refer to FIG. 7 for another embodiment of the invention. The cover layer 20 has at least two first folding lines 21 and at least two second folding lines 211 parallel with each other corresponding to the base layer 10 and extended portion 11, and a first notch 23 and a second notch 231 at each of two sides of the cover layer 20 mating each of the first and second folding lines 21 and 211. The first and second folding lines 21 and 211 and the first and second notches 23 and 231 mating the knuckles of user's finger or toe can facilitate bending or stretching of the finger or toe when in use.

As a conclusion, compared with the conventional techniques that merely cover a wound at user's finger tip or toe tip, the invention provides full coverage with the cover layer 20 on the extended portion 11 formed at a width mating the surface width of the finger or toe, and the cover layer 20 on the base layer 10 formed at a width mating the height of lateral sides of the finger or toe, thus can fully wrap the finger or toe in an annular manner. In the event that the wound is at the finger root, the base layer 10 and extended portion 11 can be lengthened, and more first and second folding lines 21 and 211 and more first and second notches 23 and 231 can be added to mate finger knuckles to wrap the finger root or toe root.

What is claimed is:

1. A full coverage adhesive patch to wrap a user's finger or toe, comprising:
a base layer which includes an extended portion at one side, a first sticking portion on a surface circumference thereof and a second sticking portion on a top edge of the extended portion; and
a cover layer located on the base layer and the extended portion on the same surface where the first and second sticking portions are located.

2. The full coverage adhesive patch of claim 1, wherein the cover layer is selectively a piece of gauze or a non-woven fabric medicated with medicine or therapeutic material.

3. The full coverage adhesive patch of claim 1, wherein the cover layer includes at least one first folding line and at least one second folding line corresponding respectively to the centers of the base layer and the extended portion, the first and second folding lines being parallel with each other.

4. The full coverage adhesive patch of claim 3, wherein the cover layer includes a first notch and a second notch at each of two sides corresponding to the first and second folding lines.

5. The full coverage adhesive patch of claim 1, wherein the cover layer is formed in a shape smaller than the profiles of the base layer and the extended portion.

6. The full coverage adhesive patch of claim 5, wherein the base layer and the cover layer are respectively formed in a symmetrical arched shape mating the shape of the finger.

7. The full coverage adhesive patch of claim 6, wherein the first sticking portion and the second sticking portion are respectively formed in a shape mating the profiles of the base layer, the extended portion and the cover layer.

8. The full coverage adhesive patch of claim 1, wherein the cover layer on the extended portion is formed at a width mating the surface width of the finger or toe; the cover layer on the base layer being formed at a width mating the height of two lateral sides of the finger or toe such that the finger or toe is fully wrapped by the cover layer in an annular fashion when in use.

9. The full coverage adhesive patch of claim 1, wherein the first sticking portion and the second sticking portion are respectively covered by a removable protective layer on the surface thereof.
